(19) United States
(12) Patent Application Publication
(10) Pub. No.: US 2023/0235852 A1
(43) Pub. Date: Jul. 27, 2023

(54) CARTRIDGE FOR STORING A NO/NITROGEN MIXTURE, AND ASSOCIATED GAS DELIVERY INSTALLATION

(71) Applicant: INOSYSTEMS, Antony (FR)

(72) Inventors: Thierry BOULANGER, Antony (FR); Frederic MARCHAL, Antony (FR)

(21) Appl. No.: 18/093,212

(22) Filed: Jan. 4, 2023

(30) Foreign Application Priority Data
Jan. 24, 2022 (FR) 2200573

(51) Int. Cl. F17C 1/14; A61M 16/10

(52) U.S. Cl. CPC F17C 1/14; A61M 16/10; F17C 2201/058; F17C 2205/0323; F17C 2221/03; F17C 2223/035; F17C 2201/0123; F17C 2203/0646; F17C 2270/025; F17C 2223/0123; A61M 2202/0275; A61M 2202/0208; A61M 2202/0266

(57) ABSTRACT

Disclosed is a cartridge for storing pressurized gas, including a main body with an internal volume for storing a gaseous mixture NO/N₂, and a distribution valve for controlling the output of the gas. The internal volume of the main body is less than 1000 ml. The concentration of NO in the gaseous mixture NO/N₂ is between 15000 and 25000 ppmv. The gas pressure in the internal volume is below 15 bar, measured at 23° C. Installation for delivering gas to a patient, including such a gas cartridge, a NO supply device fed by the gas cartridge, and a medical ventilator feeding a patient circuit which has an inhalation branch fed by the NO supply device. Use for treating patients suffering from pulmonary hypertension or hypoxia.

### CROSS-REFERENCE TO RELATED APPLICATION

This application claims priority to FR 2200573 filed Jan. 24, 2022, the entire contents of which are hereby incorporated by reference.

### BACKGROUND OF THE INVENTION

### Field of the Invention

The invention relates to a storage cartridge and its use for storing a gaseous mixture of nitric oxide and nitrogen (NO/N₂), which cartridge is designed to be fluidically connected to a device for supplying gaseous NO, serving to inject the gaseous mixture NO/N₂ into the respiratory circuit of a medical ventilator, that is to say a medical apparatus for administering gas to a patient, and also the use of such a cartridge for storing a gaseous mixture NO/N₂, and also an installation which delivers gas to a patient and comprises such a cartridge feeding a NO supply device, and a medical ventilator fed with gaseous mixture NO/N₂ by said NO supply device.

### Description of the Related Art

Nitric oxide or NO is a gas which, when inhaled, dilates the pulmonary vessels and increases oxygenation by improving gas exchange. The properties of NO are used to treat various medical conditions such as persistent pulmonary hypertension of the newborn (PPHN), acute respiratory distress syndrome (ARDS) observed mainly in adults, or pulmonary hypertension in cardiac surgery, as disclosed in particular by EP-A-560928, EP-A-1516639 or U.S. Pat. No. 10,201,564.

Generally, the NO/N₂ mixture containing a small quantity of gaseous NO (i.e. several tens or hundreds of ppm by volume) is injected, via a gaseous NO supply device, into a gaseous flow containing oxygen (O₂) circulating in the respiratory circuit of a medical ventilator and then inhaled by the patient. Typically, the gas containing oxygen is usually a mixture of N₂/O₂ or air, such as medical-grade air. Furthermore, the concentration of NO inhaled by the patient, which corresponds to a dosage, is determined by the physician or similar. Generally, the concentration of NO in the gas inhaled by the patient is between 1 and 80 ppm by volume (ppmv), depending on the population treated, i.e. neonates or adults, and therefore the disease to be treated.

Thus, U.S. Pat. No. 5,558,083 describes a NO supply device associated with a mechanical ventilator which supplies a respiratory interface delivering the NO to the patient, for example a breathing mask, a tracheal intubation tube or similar.

The NO supply device is fluidically connected to and fed by one or more gas cylinders containing the N₂/NO mixture, of which the concentration of NO is often between 200 and 1000 ppmv.

Such gas cylinders are in the form of metal cylinders or ogives, typically of steel or aluminium alloy, with a height of several tens of centimetres, generally of the order of 1 m in height, enclosing the NO/N₂ mixture at high pressure, that is to say at several tens or even hundreds of bars, for example of the order of 140 to 230 bar.

Furthermore, in order to deliver a pressure compatible with the NO supply device, these cylinders are always equipped with a pressure regulator for reducing the pressure present in the cylinder to a lower pressure, called the working pressure, at the output of the regulator. The pressure regulator is itself is metallic component, for example made of stainless steel. The cylinders of N₂/NO are therefore voluminous and/or cumbersome, and this may pose a problem when using them in emergency care units, which may be confined and already very much cluttered with other medical equipment. Moreover, the substantial volumes of gas contained in these NO/N₂ cylinders also place conditions on storage, requiring these cylinders to be held in dedicated and ventilated rooms, which poses a problem regarding space and logistics within hospital buildings.

The dimensions and the architecture of the cylinders equipped with pressure regulators, and the volumes of gas that are stored in the cylinders, inevitably result in a considerable weight, typically a weight of the order of 20 kg per cylinder. It will be appreciated that such weights lead to logistical difficulties and make the cylinders difficult for medical personnel to handle, particularly when transferring them between hospitals. Moreover, injuries have already been reported, for example when cylinders have dropped onto user's feet during cylinder replacement (i.e. when replacing an empty one with a full one), back pain after lifting, carrying or maneuvering full cylinders, etc.

Moreover, any treatment involving NO is critical, and a sudden interruption of therapy cannot be tolerated, as this would risk a rebound effect in the patient. Therefore, all NO supply devices are generally connected to two identical gas cylinders. When a cylinder runs empty, the NO supply device automatically switches to the full cylinder, which minimizes the risk of interruption but aggravates the abovementioned problem of the large space taken up in confined hospital environments, such as critical care departments.

Finally, the use of gas cylinders having a very high internal pressure, for example 140 bar or more, requires trained personnel, particularly for manipulation of the pressure regulators, which complicates the practice of NO-based therapy outside of intensive care units, for example in departments providing nursing care, but it also leads to greater logistical costs, especially as regards transporting the cylinders from the production site to the place of use, typically a hospital.

U.S. Pat. No. 10,213,572 proposes replacing the traditional gas cylinders, which are heavy and cumbersome, with a cassette enclosing a small hermetic glass vial containing liquid N₂O₄. A striker makes it possible to release the N₂O₄ by shattering the vial. Upon heating, the N₂O₄ dissociates into NO₂ which, brought into contact with solid ascorbic acid or equivalent, transforms into NO which can then be delivered in the form of NO to treat a patient. However, this type of cassette causes problems as regards logistics, storage and handling, among others, and poses risks on account of toxic chemical products being used. Moreover, their cost is high.

Other documents are also known which deal with storage of high-pressure gas, namely GB-A-2096299 and KR-A-2018/0072958, which disclose storage cartridges, in particular for carbon dioxide, FR-A-3042584, which concerns the storage of medical gases in gas containers of large dimensions, typically of up to 20 litres, compatible with very high pressures, i.e. up to 350 bar, and US-A-2018/022537, which concerns a container for an aerosol substance.

### SUMMARY OF THE INVENTION

In view of this, there is a need to be able to use NO/nitrogen mixtures in a hospital environment while avoiding all or some of the problems and disadvantages mentioned above, in particular to be able to do without traditional gas cylinders which are especially heavy and cumbersome, or cassettes whose operation involves dangerous and expensive chemical products.

A solution according to the invention therefore concerns a use of a cartridge for storing pressurized gas, comprising a main body having an internal volume of less than 1000 ml for containing the gaseous mixture, and a distribution valve for controlling the output of the gaseous mixture from the internal volume of the main body, for storing a gaseous mixture NO/N₂ having a concentration of NO of between 15000 and 25000 ppmv, at a pressure of less than 15 bar, measured at 23° C.

According to another aspect, the invention also relates to a cartridge for storing a pressurized NO/N₂ gaseous mixture, comprising:
a main body comprising an internal volume for containing the gaseous mixture, and
a gas distribution valve for controlling the output of the gaseous mixture from the internal volume of the main body,

characterized in that:
the internal volume of the main body is less than or equal to 1000 ml,
the concentration of NO in the gaseous mixture NO/N₂ is between 15000 and 25000 ppmv, and
the gaseous pressure in the internal volume of the main body is below 15 bar, measured at 23° C.

In the context of the invention:
the pressure is expressed in “bar absolute”, abbreviated to “bar”,
the volume is given in terms of water equivalent, expressed in millimetres, abbreviated to “ml”,
the contents of NO are expressed in parts per million by volume, abbreviated as “ppmv”.

Depending on the embodiment in question, the storage cartridge according to the invention and/or its use for storing the gas can comprise one or more of the following features:
the main body is made of aluminium alloy.
the main body comprises a wall having a thickness of between 0.1 and 0.5 mm.
the main body is elongate along a main axis (AA).
the main body has a height of between 15 cm and 30 cm.
the main body has an ogive shape.
the main body is closed at its upper end by a lid, preferably a lid crimped or welded onto the part of the main body of ogive shape.
the distribution valve is carried by the lid.
the distribution valve is arranged at the centre of the lid.
the main body extends between a base and an upper end, to which the lid is fixed.
the distribution valve, the lid and the main body are coaxial.
the lid is made of aluminium alloy.
the internal volume of the main body is between 500 ml and 1000 ml, preferably less than 900 ml.
the internal volume of the main body is less than or equal to 850 ml, preferably less than or equal to 800 ml.
it weighs less than 1 kg, preferably between 150 and 750 grammes.
the concentration of NO in the gaseous mixture NO/N₂ is between 15000 and 25000 ppmv, preferably at least 20000 ppmv.
the concentration of NO in the gaseous mixture NO/N₂ is between 20000 and 24000 ppmv, preferably between 22500 and 23500 ppmv, preferably at most 23000 ppmv.
the gaseous mixture NO/N₂ is formed of molecules of carbon monoxide (NO) and nitrogen molecules (N₂). However, it is not possible to rule out the presence of unavoidable impurities that result from the method of production of the gaseous mixture, in particular water vapour and/or oxygen, in negligible quantities, typically less than 3 to 5 ppmv.
the gas pressure in the internal volume of the main body is less than or equal to 13 bar, measured at 23° C., preferably less than or equal to 12 bar, more preferably less than or equal to 11.5 bar.
the gas pressure in the internal volume of the main body is at most 11.5 bar, measured at 23° C.
the main body comprises a base, an intermediate tubular portion, and an upper end closed by a lid carrying the gas distribution valve, and the intermediate tubular portion is preferably substantially of ogive shape.
the cover is crimped or welded onto the intermediate tubular portion.
the lid has a circular periphery.
the lid is configured to have a central bead, i.e. a protuberance projecting outwards, that is to say above the lid, comprising a central passage and defining an internal compartment.
the central passage of the central bead communicates with the internal compartment of said central bead.
the distribution valve comprises an escape channel associated with a seat element, said escape channel being movable in axial translation within the central passage of the central bead, and the seat element cooperating with a sealing element in order to obtain gas leaktightness and prevent any passage of gas from the internal volume of the cartridge to the escape channel.
the sealing element is a preferably flat seal, for example a flat O-ring seal.
the escape channel and the seat element are integrally joined to each other, typically fixed to each other.
the seat element is normally pushed back in the direction of the sealing element by an elastic means.
the seat element is normally pushed back in the direction of the sealing element by an elastic means, by way of a diffuser element arranged between the seat element and the sealing element.
the elastic means comes to bear on the diffuser element in order to push it back in the direction of the seat element.
the elastic element is a cylindrical helical spring.
the seat element is movable in translation in a support component forming a sleeve that accommodates the seat element and the elastic means, and preferably the diffuser element.
the support component is housed and fixed in the internal compartment of the central bead.
the escape channel comprises a free downstream end situated outside the central bead, and an upstream end situated in the internal compartment of the central bead and cooperating with the seat element.

Depending on the embodiment chosen, a gas cartridge having an internal volume of less than 900 ml is used to store the gaseous mixture NO/N₂ having a concentration of NO preferably between 20000 and 23500 ppmv, at a pressure preferably below 11.5 bar, measured at 23° C.

According to a further aspect, the invention also relates to a use of a gas cartridge according to the invention as NO source for feeding a gaseous mixture NO/N₂ to a NO supply device of an installation for delivering gas to a patient, comprising:
said at least one NO source,
said NO supply device,
an inhalation branch of a patient circuit fed with a gaseous mixture NO/N₂ by the NO supply device, and
a medical ventilator in fluidic communication with the inhalation branch in order to feed said inhalation branch with a respiratory gas containing at least 21% of oxygen, preferably air or an oxygen/nitrogen mixture.

According to a further aspect, the invention also relates to an installation for delivering gas to a patient, i.e. a human being, said installation comprising:
at least one source of NO containing a gaseous mixture NO/N₂,
a NO supply device, fed with a gaseous mixture NO/N₂ by said at least one NO source,
an inhalation branch of a patient circuit fed with a gaseous mixture NO/N₂ by the NO supply device, and
a medical ventilator in fluidic communication with the inhalation branch in order to feed said inhalation branch with a respiratory gas containing at least 21% of oxygen, preferably air or an oxygen/nitrogen mixture,

in which the NO source is a gas cartridge according to the invention having an internal volume of less than 1000 ml, preferably less than 900 ml, for storing a gaseous mixture NO/N₂ having an NO concentration of between 15000 and 25000 ppmv, typically between 20000 and 23500 ppmv at a pressure below 15 bar, typically below 11.5 bar, measured at 23° C.

According to a further aspect, the invention also relates to a method for treating a person, that is to say a patient, in particular an adult, child, adolescent or neonate, suffering from pulmonary hypertension and/or hypoxia, which can cause pulmonary vasoconstriction or similar, for example caused by one or more pulmonary diseases or disorders such as PPHN (persistent pulmonary hypertension of the newborn) or ARDS (acute respiratory distress syndrome), or those caused by heart surgery with placement of the patient on extracorporeal blood circulation, in which method a therapeutic gas containing nitrogen, oxygen and NO is administered by inhalation to the person requiring it, i.e. to the patient, said therapeutic gas being delivered by a gas delivery installation according to the invention, which comprises a medical ventilator with an inhalation branch fed with a gaseous mixture NO/N₂ coming from a gas cartridge, according to the invention, used as NO gas source.

### BRIEF DESCRIPTION OF THE DRAWINGS

The invention will now be better understood from the following detailed description, which is given by way of non-limiting illustration, with reference to the appended figures, in which:

FIG. 1 shows an embodiment of a storage cartridge used to store a NO/N₂ mixture according to the invention,

FIG. 2 shows a schematic view of an embodiment of a gas distribution valve with which the gas cartridge from FIG. 1 is equipped, shown in a closed position,

FIG. 3 shows a schematic view of the gas distribution valve from FIG. 2 in an open position,

FIG. 4 shows a schematic view of an embodiment of an installation for supplying NO to a patient, incorporating a storage cartridge used to store a NO/N₂ mixture according to the invention, such as that of FIG. 1,

FIG. 5 shows a schematic view of an embodiment of the seat element of the gas distribution valve from FIG. 2 and FIG. 3, and

FIG. 6 shows a schematic view of an embodiment of the diffuser element of the gas distribution valve from FIG. 2 and FIG. 3.

### DESCRIPTION OF THE PREFERRED EMBODIMENTS

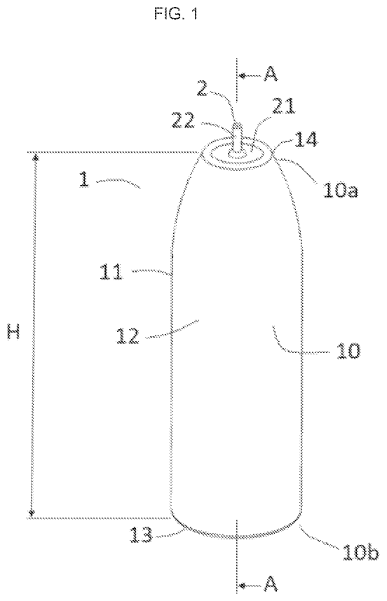

FIG. 1 shows an embodiment of a gas storage cartridge 1 used to keep, i.e. store, contain or the like, a gaseous mixture NO/N₂ according to the present invention. The gas cartridge 1 comprises a main body 11 forming a peripheral envelope defining an internal compartment or volume 12 which serves to contain and store the gaseous mixture NO/N₂.

The main body 11 is elongate along the axis AA of the cartridge 1. It has a base 13, for example a flat or curved surface, an intermediate tubular portion 10 substantially of ogive shape, and an upper end 10a closed by a lid 21 carrying a gas distribution valve 2 or dispensing valve which serves to control the output of the gaseous mixture from the internal volume 12 of the main body 11. The distribution valve 2 is equipped with an escape channel 22 for conveying the gas. The structure and the functioning of the distribution valve 2 are set out in detail below.

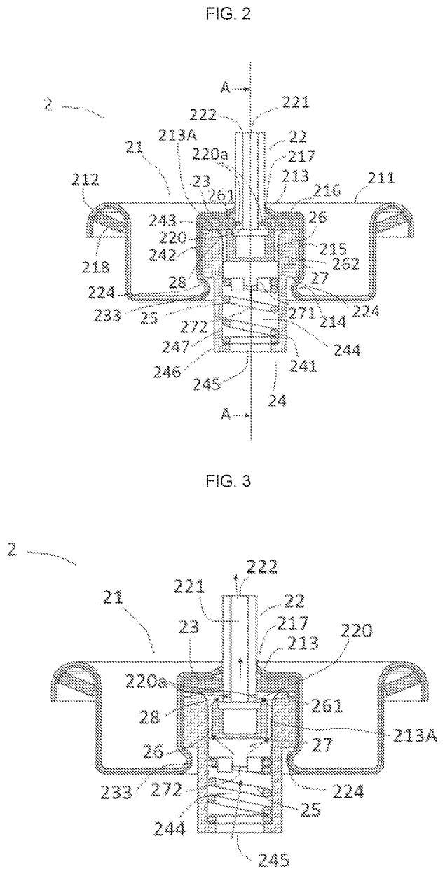

The lid 21 is preferably hermetically crimped or welded onto the perimeter 14 of the intermediate tubular portion 10, at the level of the upper end 10a thereof. In the case where the lid 21 is crimped onto the upper perimeter 14 of the main body 11, perfect sealing is obtained between them by virtue of a flat O-ring seal 218, i.e. a flat circular seal, inserted between these elements, as can be seen from FIG. 2. Moreover, the base 13 is preferably hermetically welded to the lower end 10b of the main body 11.

The main body 11 forming the peripheral envelope of the gas cartridge 1 is metallic, preferably an aluminium alloy, and has a wall with a thickness of several tenths of a millimetre, for example between approximately 0.1 and 0.5 mm. The same applies to the lid 21 and the base 13.

The gas cartridge 1 can contain, within its internal volume 12, a NO/N₂ mixture at low pressure, that is to say a pressure not exceeding one or several tens of bars, typically less than 15 bar. The internal volume 12 of the gas cartridge 1 is of limited dimensions, that is to say less than 1000 ml, preferably less than 900 ml, or even less than 800 ml, for example a volume of the order of 790 ml.

Preferably, the main body 11 of the gas cartridge 1 has a height H of between 10 cm and 30 cm, measured between the upper end 10a and the lower end 10b, i.e. at the base 13.

By virtue of its simple design and its compact dimensions, the gas cartridge 1 can be manufactured on a large scale, which is an advantage in terms of ease of production and therefore in terms of costs.

FIG. 2 and FIG. 3 show schematically the architecture and functioning of an embodiment of the gas distribution valve 2 on top of the gas cartridge 1.

The distribution valve or dispensing valve 2 is carried by the circular lid 21, which is formed by a wall or envelope 211 that is shaped, i.e. including successive specific zones or portions, in particular a rounded outer perimeter 212, a central bead 213 through which the escape channel 22 passes, and a lateral boss 214 arranged at the base of the central bead 213.

The central bead 213 forms a protuberance or bulge of cylindrical periphery protruding axially (axis AA) on the outer surface of the lid 21, being directed outwards, i.e. upwards in FIG. 1, that is to say projecting away from and above the lid 21 along the axis AA. The central bead 213 defines an internal compartment 213A. The central bead 213 is formed by deformation and/or shaping of the component forming the lid 21, such as a metal disc.

The metal lid 21, preferably of aluminium alloy, has a thickness of several tenths of a millimetre, for example from 0.1 to 0.5 mm. The central bead 213, situated at the centre of the lid 21, has a continuous recess or central passage 217, situated at its centre, housing the escape channel 22, which is in the form of a hollow duct 221. The escape channel 22 and the lid 21 are coaxial and, moreover, are likewise arranged coaxially (axis AA) on the body 11 of the gas cartridge 1.

The escape channel 22 comprises a free downstream end 222 situated on the outside, and an upstream end 220 situated in the internal compartment 213A of the central bead 213. The free downstream end 222 comprises a gas outlet orifice, while the upstream end 220 comprises lateral gas inlet channels 220a through which the gas coming from the internal volume 12 of the cartridge 1 can pass into the hollow duct 221, in order then to flow therein in the direction of the gas outlet orifice, as can be seen from FIG. 3.

At its upstream end 220, continuing or situated in the internal compartment 213A of the central bead 213 of the lid 21, the escape channel 22 comprises a seat element 26. In other words, the seat element 26 is situated in the internal compartment 213A of the central bead 213 of the lid 21. According to one embodiment, the seat element 26 is here in the form of a cylindrical body 261 comprising a central recess 263 that has a closed base.

The escape channel 22 is rigidly connected to the seat element 26 by, for example, force-fit engagement on a shoulder 262 by welding or any other technique. According to another embodiment, the escape channel 22 and the seat element 26 can be formed in one piece.

The escape channel 22 is able to move in translation, in the central passage of the central bead 213 of the lid 21, in the direction of the internal compartment 213A of the central bead 213. The escape channel 22 and the seat element 26 thus form an assembly or system which controls the output/release of gas and which is axially movable (axis AA). In fact, when it is in contact with a sealing element such as a flat O-ring seal 23 arranged in the bottom 216 of the central bead 213, the front face 261 of the seat element 26 ensures fluidic leaktightness between the seat element 26 and the sealing element 23, in order then to prevent any output of gas from the cartridge 1, as is illustrated in FIG. 2 and set out in detail below. Conversely, when the front face 261 of the seat element 26 is no longer in contact with the sealing element 23, a space is created between them, that is to say an interruption in leaktightness, which allows the gas to pass through and enter the hollow duct 221 and then flow there in the direction of the gas outlet orifice.

Moreover, a tubular support component 24, preferably of cylindrical shape, forms a sleeve 241 having an outer wall 242, which is itself cylindrical. The support component 24 is inserted into the internal compartment 213A of the central bead 213 formed in the envelope 211 of the lid 21, that is to say in the internal part of the central bead 213 of the lid 21.

More precisely, part of the outer wall 242 of the sleeve 241 comes into contact with an internal lateral portion 215 of the central bead 213 of the lid 21. The support component 24 is then held in position by a lateral boss 214 which is obtained by deformation, oriented radially towards the axis AA, that is to say towards the inside of the internal compartment 213A of the central bead 213, on a part of the envelope 211 or wall situated at the base of the central bead 231 of the lid 21.

In other words, at its base 233, the central bead 213 comprises a lateral boss 214 by which it is possible to maintain the support component 24 in a fixed position in the internal compartment 213A of the central bead 213, by cooperating with a shoulder 224 situated on the outer wall of the tubular support component 24. The lateral boss 214 corresponds to an indentation or a deformation of the peripheral wall of the central bead 213 (at its base 233) directed towards the inside of the internal compartment 213A of the central bead 213, which is to say that the inner wall of the internal compartment 213A of the bead 213 forms, at its base 233, an annular expansion extending into the internal compartment 213A and cooperating with the shoulder 224 situated on the outer wall of the tubular support component 24.

The tubular support component 24 thus forms a sleeve around the seat element 26 situated at the inner end of the escape channel 22, such that, during the translational movements of the escape channel 22 in the central passage of the central bead 213 of the lid 21, said seat element 26, which is integral with the escape channel 22, is itself also able to move in translation at the same time within the sleeve formed by the tubular support component 24, that is to say in the housing or internal volume 244 situated at the centre of the tubular support component 24, as can be seen from FIG. 3.

Moreover, the support component 24 comprises an annular end 243 that comes to bear on a sealing element, such as a flat O-ring (i.e. annular) seal 23, arranged in the internal base 216 of the central bead 213 and passed through coaxially by the escape channel 22. In other words, the annular end 243 of the support component 24 is also coaxial with the escape channel 22 and with the O-ring seal 23, i.e. the sealing element, such that the O-ring seal 23 is sandwiched and compressed between the annular end 243 of the support component 24 and the internal base 216 of the central bead 213 of the lid 21.

The sealing element, such as a flat O-ring seal 23, is thus held in position by the support component 24 forming a sleeve, whilst the escape channel 22 is able to slide at the recessed centre of the sealing element, i.e. the ring-shaped seal.

Moreover, the seat element 26 is continued by a diffuser element 27, which itself is also able to slide in the internal volume 244 of the support component 24.

This diffuser element 27 is of substantially cylindrical shape and has a neck 271 traversed, at its centre, by a central channel 272. The central channel 272 then forms a fluidic connection between the internal volume 244 of the support component 24 and an internal chamber 28 situated, on the one hand, between the outer surface 262 of the seat element 26 and the inner surface 247 of the support component 24 and, on the other hand, between the O-ring seal 23 and the diffuser element 27.

As can be seen from FIG. 2 and FIG. 3, the diffuser element 27 is also located in the internal compartment 213A of the central bead 213 of the lid 21.

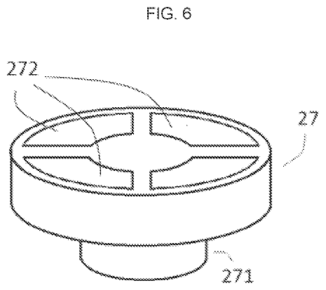

According to one embodiment, the diffuser element 27 can be in the form of a tubular structure with a neck 271 and openings 272 for the gas, as is illustrated in FIG. 6. The neck 271 of the diffuser element 27, which is oriented towards the internal volume 12 of the cartridge 1, that is to say which projects axially (AA) towards the inside of the cartridge 1, is able to receive and hold in position one of the ends of an elastic means 25, here a spring of cylindrical shape, which is housed and compressed between the diffuser element 27 and the base 246 of the support component 24.

The spring 25 normally pushes the diffuser element 27 back in the direction of the seat element 26 in such a way as to ensure fluidic leaktightness between said seat element 26 and the O-ring seal 23, as is illustrated in FIG. 2.

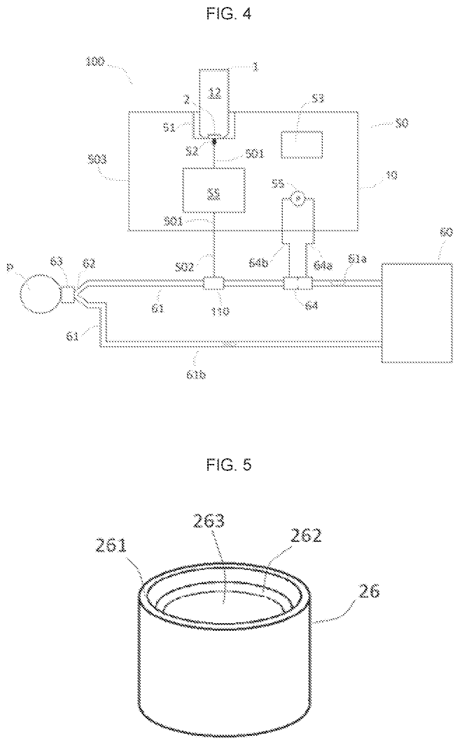

The base 246 of the support component 24 is moreover traversed by an axial channel or passage 245. There is therefore a fluidic communication between the axial passage 245 and the internal volume 244 of the support component 24, the central channel 272 of the diffuser element 27 and the internal chamber 28 allowing the gaseous flow, i.e the gaseous mixture NO/N₂, to move through these elements before leaving the internal volume 12 of the cartridge 1 when the gas is being used, i.e. sent to a respiratory circuit 61 of a ventilator 60 via a NO supply device 50, as is shown schematically in FIG. 4.

More precisely, in what is called the “closed” or “at rest” configuration as illustrated in FIG. 2, the spring 25 pushes the diffuser element 27, and consequently also the seat element 26, in such a way that the front face 261 of said seat element 26 comes to compress the flat O-ring seal 23 and thus ensures perfect fluidic leaktightness between the internal chamber 28 of the support component 24 and the hollow internal duct 221 of the escape channel 22.

In this “closed” configuration, no gas is supplied via the hollow internal duct 221 of the escape channel 22. In other words, the gas contained in the internal volume 12 of the gas cartridge 1 is in fluidic communication with the axial channel or passage 245, and the internal volume 244 of the support component 24, and diffuses as far as the internal chamber 28 but cannot escape into the hollow internal duct 221 of the escape channel 22 via the lateral gas inlet channels 220a.

Conversely, FIG. 3 illustrates the valve 2 in what is called the “open” position, in which gas is supplied via the hollow internal duct 221 of the escape channel 22.

In order to change the valve 2 from the “closed” configuration in FIG. 2 to the “open” configuration in FIG. 3, it suffices to apply to the free end 222 of the escape channel 22 a mechanical force that forces said escape channel 22 to execute an axial translation along the axis AA in the direction of the cartridge 1, in such a way that the escape channel 22 slides in the central passages of the central bead 213 of the lid 21 and of the flat O-ring seal 23.

In other words, applying an external force to the free end 222 of the escape channel 22, in order to push the tubular escape channel 22 towards the valve 2 and the cartridge 1, results in a release of the gas, which can then pass into the lumen or hollow internal duct 221 of the escape channel 22 and then exit the latter via the outlet orifice which is situated at the free end 222 of the escape channel 22.

The external force applied to the end 222 of the escape channel 22 can result from the insertion of the cartridge 1 into a specific housing 51 formed in the NO supply device 50, which specific housing 51 comprises an actuation mechanism 52 (not shown in detail) for cooperating with the gas distribution valve 2 of the gas cartridge 1 according to the invention, as shown schematically in FIG. 4, in order to release the gas.

On account of the coupling existing between the escape channel 22 and the seat element 26, the seat element 26 also executes an axial translation movement along the axis AA and then simultaneously pushes the diffuser element 27 back, generating a more pronounced compression of the cylindrical helical spring 25, as can be seen from FIG. 3.

In the process, the front face 261 of the seat element 26 then loses contact with the flat O-ring seal 23 (which remains in position, as in FIG. 2), which generates a loss of fluidic leaktightness between them.

The internal chamber 28 is then in fluidic communication not only with the hollow internal duct 221 via the lateral gas inlet channels 220a of the escape channel 22, but also with the central channel 272 of the diffuser element 27, the internal volume 244 and the channel 245 of the support component 24. A circulation of gas can then be established through these different elements and in the direction of the gas outlet orifice carried by the free end 222 of the escape channel 22, as is illustrated in FIG. 3.

In FIG. 3, the arrows indicate the circulation of gas from the internal volume 12 of the cartridge 1 and through the distribution valve 2.

The gas contained in the internal volume 12 of the gas cartridge 1, that is to say the NO/N₂ mixture, then diffuses via the internal chamber 28 as far as the hollow internal duct 221 of the escape channel 22, through which it can then escape and be collected by the internal circuit 501 of the NO supply device 50, as is explained below and illustrated in FIG. 4.

In other words, the gas cartridge 1 provided with such a distribution valve 2 is preferably intended, according to the invention, to be inserted in a dedicated housing 51 of a NO supply device 50 and to be maintained in position there in the “open” configuration of the valve 2 via a mechanical stress applied to the free end 222 of the escape channel 22, making it possible to push this back in translation and thereby release the gas, i.e. the NO/N₂ mixture, contained in the cartridge 1.

By way of example, a gas cartridge is generally designed having an internal volume 12 of the order of 790 ml enclosing a binary gas mixture N₂/NO pressurized to about 10 bar measured at about 23° C., said N₂/NO mixture containing about 23000 ppmv of NO, the remainder being nitrogen, and possibly a negligible quantity of unavoidable impurities such as water vapour or gaseous oxygen.

This is equivalent to 7900 ml of a gaseous mixture N₂/NO at atmospheric pressure (i.e. 1 bar) and additionally corresponds to a volume of NO, thus stored, of about 180 ml for the considered content of 23000 ppmv of NO.

By comparison, a gas cylinder traditionally used to store N₂/NO mixtures is able to deliver 1963 l of a N₂/NO mixture having a NO concentration of 800 ppmv. This corresponds to an available volume of NO of 1570 ml, i.e. a ratio of the available NO volume of about 10.

The gas cartridge 1 according to the invention offers greater ease of use and straightforward logistics, has a smaller overall size and, during use, poses virtually zero risk (injuries if dropped or when manipulating dangerous products) to the operators, that is to say healthcare personnel, and it can be manufactured more simply and therefore at a very much lower cost.

Moreover, a gas cartridge 1 according to the invention also meets the requirements and regulations governing the transportation and shipment of compressed gas, and therefore this type of cartridge 1 can be carried by a public courier or delivery service such as Fedex or UPS. This also has an undeniable advantage in terms of logistics and ease of operation.

FIG. 4 shows a schematic view of an embodiment of an installation 100 by which a gas containing NO is supplied or delivered to a patient, said installation incorporating a gas storage cartridge 1 used to store a gaseous mixture NO/N₂ according to the invention, for example the storage cartridge 1 from FIG. 1. This installation 100 for delivering gas to a patient P comprises the gas cartridge 1 serving as source of a gaseous mixture NO/N₂ containing between 15000 and 25000 pppmv of NO (the remainder being nitrogen) according to the invention, arranged in such a way as to feed, via its gas distribution valve 2, the upstream portion of the internal gas circuit 501 of a NO supply device 50 designed to deliver the gaseous mixture NO/N₂ to the respiratory circuit 61 of a medical ventilator 60.

The gas cartridge 1 used according to the invention comes preferably to be accommodated in a dedicated housing 51 of the NO supply device 50 comprising an actuation mechanism 52 (not shown in detail) for cooperating with the distribution valve 2 of the gas cartridge 1 according to the invention, in particular by bearing on the end 222 of the escape channel 22 so as to push the tubular escape channel 22 back in the direction of the cartridge 1 and thereby permit the passage of the gas from the internal volume 12 of the gas cartridge 1 according to the invention to said internal gas circuit 501 of the NO supply device 50.

According to another embodiment, two or more identical gas cartridges 1 can be inserted in the dedicated housing 51 of the NO supply device 50 so as to allow switching to a full cartridge 1 as and when the other cartridge 1, which is in use, begins to run empty, thereby ensuring continuity of the therapy, that is to say avoiding any interruption in the delivery of NO to the patient P. In other words, in this case, the two gas cartridges 1 are arranged in parallel with each other, in such a way as to be used alternately. The empty cartridge can then be replaced, while the other supplies gas, i.e. the NO/N₂ mixture. In this case, the dedicated housing 51 of the NO supply device 50 is dimensioned to accommodate several cartridges 1 and moreover comprises a dedicated actuation mechanism 52 for each cartridge, that is to say actuation mechanisms 52 that come to cooperate with the gas distribution valve 2 provided on each gas cartridge 1.

In all cases, the fluidic connection of the one or more gas cartridges 1 to the NO supply device 50 is made leaktight by the use of sealing means such as O-ring seals or the like.

In the NO supply device 50, control means 55 are provided for controlling the flow rate and/or the pressure of the gas, these means being arranged on the internal gas circuit 501, making it possible to control or adjust the flow rate and/or the pressure of the gas conveyed through the internal gas circuit 501 of the NO supply device 50, for example a pressure regulator, one or more control valves, for example solenoid valves, one or more calibrated orifices, one or more non-return valves, etc.

The means 55 for controlling the flow rate and/or the pressure of the gas are operated by operating means 53, also called an operating unit, such as an electronic card with microprocessor(s) using one or more algorithms, or any other suitable control system.

The gaseous flow of NO/N₂, output by the control means 55 for controlling the flow rate and/or the pressure of the gas, is conveyed through the downstream portion of the internal gas circuit 501, then through an injection duct 502, before being fed into the inhalation branch 61A of the respiratory circuit 61 connected fluidically to a medical ventilator 60, namely a respiratory assistance device delivering a respiratory gas containing at least 21% of oxygen, typically air or a nitrogen/oxygen mixture.

To facilitate understanding, the respiratory gas delivered by the medical ventilator 60 is considered to be air. This air circulates through the inhalation branch 61A from the ventilator 60 as far as a respiratory interface 63, such as a mask or a tracheal tube, supplying the patient P with the therapeutic mixture containing the NO in the desired content.

The gaseous flow of NO/N₂ passed through the injection conduit 502 mixes with the air directly in the inhalation branch 61A of the respiratory circuit 61 in such a way as to obtain a final therapeutic mixture containing essentially oxygen, nitrogen and the desired proportion of NO.

The desired proportion of NO depends on the dosage fixed by the physician, the type of patient (adult, child, neonate. etc.), the pathology in question (PPHN, pulmonary hypertension, etc.), or other considerations. Generally, the content of NO is between 1 and 80 ppmv of NO in the final therapeutic mixture delivered to the patient P via the respiratory interface 63, that is to say a final gaseous mixture containing essentially oxygen, nitrogen and NO.

The respiratory circuit 61 for the patient additionally comprises an exhalation branch 61B for recovering the CO₂-rich gases exhaled by the patient and for conveying them to the medical ventilator 1, in particular for analysis purposes, before they are released to the atmosphere.

The inhalation branch 61A and the exhalation branch 61B, for example flexible hoses, are attached to a connection piece 62, called a Y connection, arranged upstream from the respiratory interface 63.

A gas humidifier (not shown) can optionally be arranged on the inhalation branch 61A in order to humidify the gas delivered to the patient P.

Moreover, a flow rate sensor 64 is arranged on the inhalation branch 61A, between the ventilator 60 and the site of injection of NO delivered by the injection conduit 52. This flow rate sensor 64 can comprise an upstream line 64A and a downstream line 64B for pressure measurement, which lines are fluidically connected to the flow rate sensor 64 at connection sites situated upstream and downstream from an internal restriction, in order to carry out the pressure measurements there on the circulating flow, before and after head loss caused by the internal restriction (not shown in detail).

The lines 64A, 64B form pressure measurement ducts which convey the measurements of the pressure of the circulating flow, before and after head loss, to a differential pressure sensor 55 arranged in the NO supply device 50. This differential pressure sensor 55 is integrated in the housing 503 of the NO supply device 50 and is either connected electrically to the operating unit 53 or transmits the pressure measurements to the latter so that they are processed by computer.

The operating unit 53 additionally constitutes a data processing system, in particular of the measurements carried out by the sensors or others, making it possible in particular to determine the flow rate of air circulating in the inhalation branch 61A.

Knowing this flow rate allows the operating means 53 to determine the quantity of NO/N₂ to inject into the air flow in order to obtain the desired content of NO in the final therapeutic mixture, that is to say resulting from the injection of the NO/N₂ mixture into the air circulating in the lumen of the inhalation branch 61A. The operating means 53 will then cooperate with the means 55 for controlling the flow rate and/or pressure of the NO supply device 50 so as to regulate the flow of NO/N₂ mixture (i.e. flow rate and/or pressure) fed to the injection circuit 502 supplying the NO/N₂ mixture.

By using a gas cartridge 1 according to the invention within such an installation 100 for supplying, i.e. delivering, a gas containing NO to a patient, it is possible to reduce the overall size of the installation while facilitating the manipulation of the latter, in particular the replacements of the empty cartridge by a full cartridge 1, all of this at a controlled cost.

Such an installation can be used to deliver gaseous mixtures based on NO to patients, in particular adults, children, adolescents or neonates, suffering from pulmonary hypertension and/or hypoxia, which can cause pulmonary vasoconstriction or similar, for example caused by pulmonary diseases or disorders such as PPHN (persistent pulmonary hypertension of the newborn) or ARDS (acute respiratory distress syndrome), or those caused by heart surgery with placement of the patient on extracorporeal blood circulation.

1. A cartridge for storing pressurized gas, comprising:
a main body comprising an internal volume of less than 1000 ml for containing the gaseous mixture, and
a distribution valve for controlling the output of the gaseous mixture from the internal volume of the main body,
for storing a gaseous mixture NO/N₂ having a concentration of NO of between 15000 and 25000 ppmv, at a pressure of less than 15 bar, measured at 23° C.

2. The cartridge of claim 1, wherein the internal volume of the main body of the cartridge is less than or equal to 900 ml.

3. The cartridge of claim 1, wherein the concentration of NO in the gaseous mixture NO/N₂ is between 20000 and 24000 ppmv.

4. The cartridge of claim 1, wherein the gas pressure in the internal volume of the main body is less than or equal to 13 bar, measured at 23° C.

5. The cartridge of claim 1, wherein the main body has an ogive shape and/or is made of aluminum alloy.

6. The cartridge of claim 1, wherein the main body of the cartridge is closed at an upper end by a lid, the distribution valve being carried by the lid.

7. The cartridge of claim 6, wherein:
the lid of the cartridge is configured to have a central bead comprising a central passage and defining an internal compartment, and
the distribution valve of the cartridge comprises an escape channel associated with a seat element, said escape channel being movable in axial translation within the central passage of the central bead, and the seat element cooperating with a sealing element in order to obtain gas leaktightness and prevent any passage of gas from the internal volume of the cartridge to the escape channel.

8. The cartridge of claim 7, wherein the seat element is normally pushed back in the direction of the sealing element by an elastic means.

9. The cartridge of claim 7, wherein the seat element is movable in translation in a support component forming a sleeve housing the seat element and the elastic means, said support component being housed and fixed in the internal compartment of the central bead.

10. The cartridge of claim 7, wherein the escape channel comprises a free downstream end situated outside the central bead, and an upstream end situated in the internal compartment of the central bead and cooperating with the seat element.

11. The cartridge of claim 2, wherein the internal volume of the main body of the cartridge is less than or equal to 800 ml.

12. The cartridge of claim 3, wherein the concentration of NO in the gaseous mixture NO/N₂ is between 22500 and 23500 ppmv.

13. The cartridge of claim 1, wherein the internal volume of the main body is at least 500 ml.

14. An installation for delivering gas to a patient, comprising:
at least one NO source comprising the gas cartridge of claim 1,
an NO supply device supplied with a gaseous mixture NO/N₂, wherein the NO is supplied by the gas cartridge,
an inhalation branch of as patient circuit fed with the gaseous mixture NO/N₂ by the NO supply device, and
a medical ventilator in fluidic communication with the inhalation branch in order to feed said inhalation branch with a respiratory gas containing at least 21% of oxygen.

15. The installation of claim 14, wherein the respiratory gas containing at least 21% of oxygen is air or an oxygen/nitrogen mixture.

16. The cartridge of claim 4, wherein the gas pressure in the internal volume of the main body is less than or equal to 12 bar, measured at 23° C.

17. The cartridge of claim 4, wherein the gas pressure in the internal volume of the main body is less than or equal to 11.5 bar, measured at 23° C.

18. The cartridge of claim 6, wherein the lid comprises aluminum alloy.

19. The cartridge of claim 7, wherein the seat element is normally pushed back in the direction of the sealing element by an elastic means byway of a diffuser element arranged between the seat element and the sealing element.

20. A process for storing a pressurized gas in a cartridge, comprising:
a) providing a cartridge comprising a main body comprising an internal volume of less than 1000 ml for containing a gaseous mixture, and a distribution valve for controlling the output of the gaseous mixture from the internal volume of the main body, and
b) storing a gaseous mixture NO/N₂ comprising a concentration of NO of between 15000 and 25000 ppmv, at a pressure of less than 15 bar, measured at 23° C., in the internal volume of said cartridge.
